(19) United States
(12) Patent Application Publication
(10) Pub. No.: US 2025/0362208 A1
(43) Pub. Date: Nov. 27, 2025

(54) RIGID CHAIN PROBE SYSTEM FOR GRAIN SAMPLING

(71) Applicant: HRSW LLC, Alden, IA (US)

(72) Inventors: Jerry Hartema, Alden, IA (US); Richard George Struve, Gilbert, IA (US)

(21) Appl. No.: 19/219,289

(22) Filed: May 27, 2025

Related U.S. Application Data
(60) Provisional application No. 63/651,772, filed on May 24, 2024.

(51) Int. Cl. G01N 1/08; G01N 33/02

(52) U.S. Cl. CPC G01N 1/08; G01N 33/02

(57) ABSTRACT

A probe structure for driving a probe into granulate is described within. The probe structure includes a chassis with a lower frame, a midframe, a horizontal rail, and a probe opening. The structure has a reel assembly with a reel sprocket on the horizontal rail, a drive sprocket, a motor coupled to the drive sprocket. The probe support is disposed around the reel assembly and has interlocking chain links with an anti-rotation protrusion, a rotation indentation, and a drive pin extending across a width of the chain link to engage the drive wheel. The probe is located within a central space of each of the plurality of chain links.

### CROSS-REFERENCE TO RELATED APPLICATION(S)

This application claims the benefit under 35 U.S.C. § 119 (e) to U.S. Provisional Application 63/651,772, filed May 24, 2024 and entitled “Rigid Chain Probe System for Grain Sampling,” which is hereby incorporated herein by reference in its entirety.

### TECHNICAL FIELD

The disclosure relates to grain processing, and more particularly, a system for autonomously processing grain entering a facility using a probe that is strong enough to plunge into grain and flexible to allow for unintended movement of the grain while embedded within.

### BACKGROUND

Grain facilities currently require regular sampling of any grain entering a facility. In order to ensure random sampling of the grain stored in a trailer of a truck, automatic sampling devices may be used. These automatic sampling devices typically drive a probe into the grain at a randomly predetermined location of the trailer. From time to time, a driver of the grain truck may sometimes pull forward or reverse while the probe is still embedded within the grain of the trailer, which breaks the probe and leads to much downtime while the probe assembly is refitted to the system.

### SUMMARY

In Example 1, the disclosure includes a probe structure for driving a probe into granulate. The probe structure includes a chassis with a lower frame, a midframe, a horizontal rail, and a probe opening. The structure has a reel assembly with a reel sprocket on the horizontal rail, a drive sprocket, a motor coupled to the drive sprocket. The probe support is disposed around the reel assembly and has interlocking chain links with an anti-rotation protrusion, a rotation indentation, and a drive pin extending across a width of the chain link to engage the drive wheel. The probe is located within a central space of each of the plurality of chain links.

Example 2 relates to the probe structure of Example 1, wherein the anti-rotation protrusion and the rotation indentation cooperate to allow rotation of a first of the plurality of chain links with respect to a second of the plurality of chain links in one direction.

Example 3 relates to the probe structure of Example 2, wherein the anti-rotation protrusion and the rotation indentation cooperate to prevent rotation of a first of the plurality of chain links with respect to a second of the plurality of chain links in a second direction opposite the first direction.

Example 4 relates to the probe structure of Example 1, wherein each chain link comprises a pair of drive pins disposed on an inner portion of the chain link and substantially parallel to one another.

Example 5 relates to the probe structure of Example 1, further comprising at least one linkage connecting the at least one drive pin of a first chain link to the at least one drive pin of a second chain link adjacent to the first chain link.

Example 6 relates to the probe structure of Example 5, wherein the at least one linkage comprises a pair of linkages connecting a first drive pin of a first chain link to a second drive pin of a second chain link adjacent to the first chain link.

Example 7 relates to the probe structure of Example 1, wherein the tensioning strap is configured to urge the anti-rotation protrusion of a first chain link and the rotation indentation of a second adjacent chain link together.

Example 8 relates to the probe structure of Example 1, wherein the probe support further comprises a tensioning strap, and wherein the tensioning strap is configured to urge the anti-rotation protrusion of a first chain link and the rotation indentation of a second adjacent chain link together, thereby providing a stiffening force to a vertical probing portion of the probe support.

Example 9 relates to the probe structure of Example 1, wherein the chassis is configured to attach to a subframe of the mainframe, the subframe selectively movable in at least one of a fore/aft direction and a lateral direction of the mainframe.

Example 10 relates to the probe structure of Example 1, wherein the probe support comprises a fully extended position wherein the probe support reaches downwardly substantially to a floor of a container, and a fully retracted position wherein the probe support is substantially contained within the chassis.

Example 11 relates to the probe structure of Example 1, wherein the probe support further comprises a tensioning strap disposed on an outside of each of the plurality of chain links, the tensioning strap attached at a first end to the tensioning plate and at a second end to a chain link most distal from the tensioning plate.

Example 12 relates to the probe structure of Example 1, wherein the chassis further comprises a horizontal rail and the reel assembly further comprises a reel sprocket slidably coupled to the horizontal rail.

Example 13 relates to the probe structure of Example 1, wherein the probe support comprises a fully extended position wherein the reel sprocket is adjacent the drive sprocket and the probe support reaches downwardly substantially to a floor of a container, and a fully retracted position wherein the reel sprocket is in a position most distal from the drive sprocket and the probe support is substantially contained within the chassis.

In Example 14, the disclosure is directed to a probe reel assembly configured to provide support for driving a probe into granulate and allow flexion of the probe in one direction. The probe reel includes a chassis configured to attach to a mainframe. The chassis has a lower frame with a probe opening and a midframe above the lower frame. A drive sprocket is above the probe opening and is attached to a motor and a reel sprocket slidably coupled to the chassis. A probe support assembly includes interlocking chain links around and operably connected to the reel sprocket and the drive sprocket. Each of the plurality of chain links includes an anti-rotation protrusion, a rotation indentation on an opposite side of the chain link from the anti-rotation protrusion, and at least one drive pin extending across a width of the chain link and is configured to engage the drive wheel. The rotation indentation of each chain link is configured to accept the anti-rotation protrusion from an adjacent chain link. The probe is within a central space of each of the plurality of chain links and extends from a tensioning plate on the chassis at a first end and through the link most distal from the tensioning plate at a second end.

Example 15 relates to the probe reel of Example 14, further comprising a horizontal rail proximate the midframe, and a reel sprocket slidably coupled to the horizontal rail.

Example 16 relates to the probe reel of Example 15, wherein the horizontal rail is disposed on the chassis.

Example 17 relates to the probe reel of Example 15, wherein the reel sprocket is disposed on the horizontal rail.

Example 18 relates to the probe reel of Example 14, further comprising a tensioning strap disposed on an outside of each of the plurality of chain links, the tensioning strap attached at a first end to the tensioning plate and at a second end to a chain link most distal from the tensioning plate.

In Example 19, the disclosure is directed to a granulate probing system. The system includes a mainframe to allow a tractor trailer to drive underneath an upper frame portion, a subframe on the upper frame portion, the subframe movable in a fore/aft and lateral directions on the upper frame portion, and a chassis disposed on the subframe. The subframe includes a lower frame, a midframe substantially horizontally above the lower frame, and a horizontal rail proximate the midframe. The system includes a reel assembly having a reel sprocket slidably coupled to the horizontal rail, a drive sprocket having an edge disposed above the probe opening, and a motor on the chassis and operably coupled to the drive sprocket. The system has a probe support assembly around the reel sprocket and the drive sprocket, and having a plurality of chain links cooperating in an interlocking manner to form the probe support assembly. Each chain link has an anti-rotation protrusion, a rotation indentation on an opposite side of the chain link from the anti-rotation protrusion and configured to accept the anti-rotation protrusion from an adjacent chain link; and at least one drive pin extending across a width of the chain link and configured to engage the drive wheel such that movement of the drive wheel drives the probe support. the system includes a probe disposed within a central space of each of the plurality of chain links and extending from a tensioning plate disposed on the chassis at a first end and through the link most distal from the tensioning plate at a second end.

Example 20 relates to the granulate probing system of Example 19, wherein the probe support further comprises a tensioning strap disposed on an outside of each of the plurality of chain links, the tensioning strap attached at a first end to the tensioning plate and at a second end to a chain link most distal from the tensioning plate.

In another example, the disclosure is directed to an apparatus comprising means for performing any of the techniques described herein.

In another example, the disclosure is directed to a method comprising any of the techniques described herein.

In another example, the disclosure is directed to any of the techniques described herein.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present disclosure and therefore do not limit the scope of the invention. The drawings are not necessarily to scale, though examples can include the scale illustrated, and are intended for use in conjunction with the explanations in the following detailed description wherein like reference characters denote like elements. Examples of the present disclosure will hereinafter be described in conjunction with the appended drawings.

FIG. 1A is a conceptual diagram illustrating an example rigid chain probe system, in accordance with the techniques described herein.

FIG. 1B is a top-down view of a conceptual diagram illustrating an example rigid chain probe system, in accordance with the techniques described herein.

FIG. 2 is a conceptual diagram of a subframe of an embodiment of the example rigid chain probe system, in accordance with the techniques described herein.

FIG. 3A is a conceptual diagram of a chassis and reel system of an embodiment of the example rigid chain probe system, in accordance with the techniques described herein.

FIG. 3B is a side view of a conceptual diagram of a chassis and reel system of an embodiment of the example rigid chain probe system, in accordance with the techniques described herein.

FIG. 3C is another side view of a conceptual diagram of a chassis and reel system of an embodiment of the example rigid chain probe system, in accordance with the techniques described herein.

FIG. 4A is a conceptual diagram of a probe of an embodiment of the example rigid chain probe system, in accordance with the techniques described herein.

FIG. 4B is another conceptual diagram of a probe of an embodiment of the example rigid chain probe system, in accordance with the techniques described herein.

FIG. 4C is a conceptual diagram of a chain link of an embodiment of the example rigid chain probe system, in accordance with the techniques described herein.

FIG. 4D is a conceptual diagram of a linkage of an embodiment of the example rigid chain probe system, in accordance with the techniques described herein.

FIG. 4E is yet another conceptual diagram of a probe of an embodiment of the example rigid chain probe system, in accordance with the techniques described herein.

FIG. 4F is a conceptual diagram of a tensioning system of an embodiment of the example rigid chain probe system, in accordance with the techniques described herein.

FIG. 4G is another conceptual diagram of a tensioning system of an embodiment of the example rigid chain probe system, in accordance with the techniques described herein.

### DETAILED DESCRIPTION

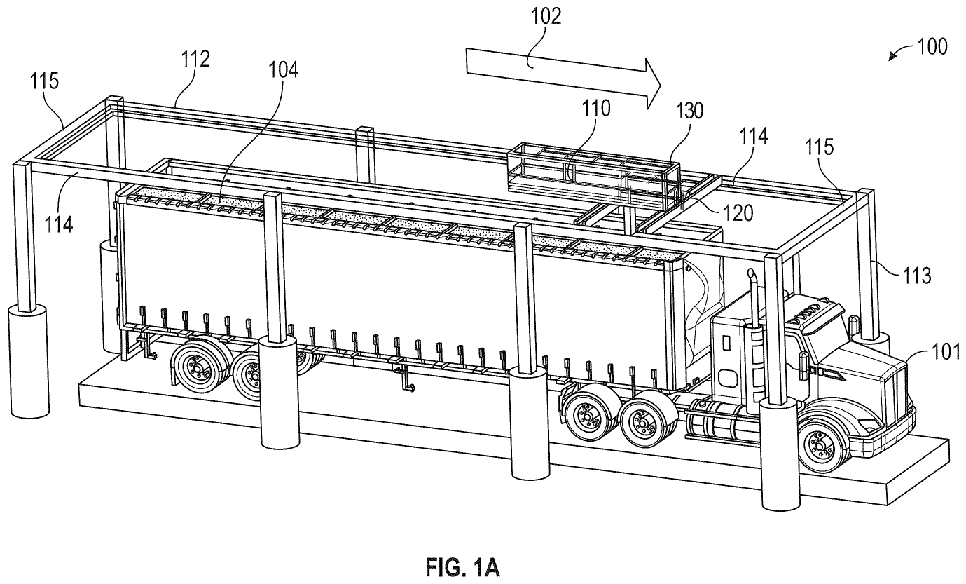

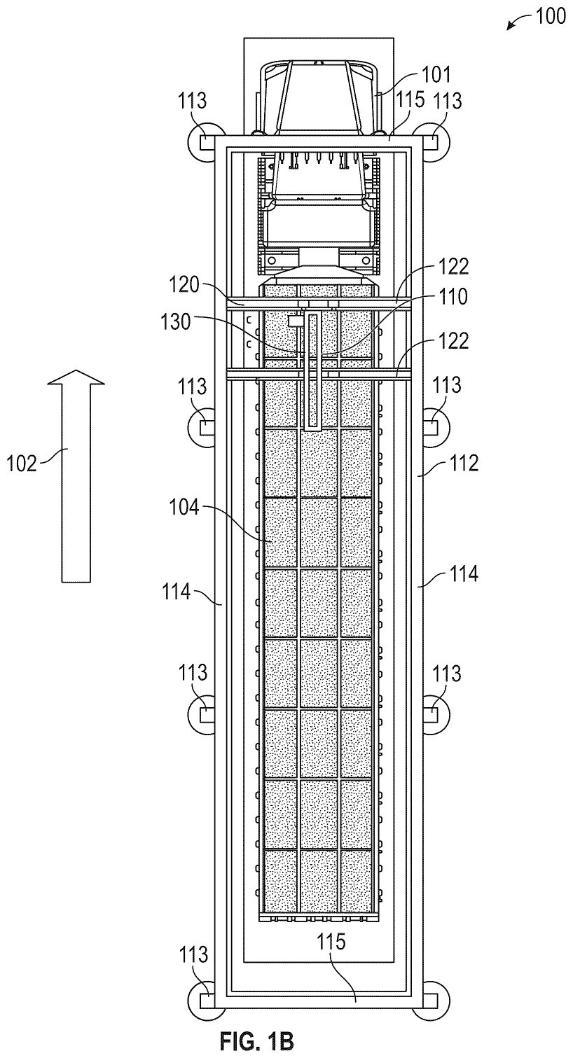

The following detailed description is exemplary in nature and is not intended to limit the scope, applicability, or configuration of the techniques or systems described herein in any way. Rather, the following description provides some practical illustrations for implementing examples of the techniques or systems described herein. Those skilled in the art will recognize that many of the noted examples have a variety of suitable alternatives. As used throughout this disclosure, fore/aft is in the direction of the arrow 102 on FIG. 1B, side to side or lateral is right to left or vice versa on FIG. 1B, and up/down is in and out of the page as shown in FIG. 1B.

FIGS. 1A and 1B show various components of an embodiment of a grain processing facility 100. The grain processing facility 100 may be a gantry-type crane system that allows for the positioning of a probe in a desired location of a trailer of a truck 101 that has driven within the facility 100. In the example of FIG. 1, truck 101 carries grain 104 into autonomous grain processing facility 100, typically in the direction of arrow 102. Truck 101 may be any truck with a cargo compartment that is at least partially open on a top portion of the cargo compartment such that the cargo compartment can carry grain 104 and that grain 104 can be drawn up by probe system 110 above grain 104 to be evaluated. The probe system in the embodiments described below is a rigid chain probe system 110 and may be called as such throughout the disclosure. The grain 104 may be any agricultural plant product that can be sampled or evaluated, including wheat, corn, soybeans, hops, rice, oats, cornmeal, barley, cannabis, or any other crop that could be sampled and evaluated using the techniques described herein.

In some embodiments, the truck 101 may enter autonomous grain processing facility 100 with grain 104 and park within the bounds of a mainframe 112. In some embodiments, a subframe 120 is attached to the mainframe 112. As described in more detail below, the subframe 120 may include crossarms 122, slidable beams, one or more motors, a support plate, and any other attachment and movement elements that allow the subframe to move both laterally and fore/aft to carry the probe system 110 to a desired location within a trailer of the truck 101.

In some embodiments, the probe system 110 includes a chassis 130. The chassis 130 may be any size, shape, and configuration suitable to slidingly (e.g., movable in a left/right direction in any manner known in the art) attach to the subframe 120 and provide a stable platform for the probe system 110. In some embodiments, the chassis 130 is disposed on a top side of the subframe 120 such that a truck 101 is able to drive under the probe system 110 and subframe when the probe of the probe system 110 is in the retracted position (as described in more detail below). The subframe 120 may then be moved forward or rearward to locate the probe system 110 in the desired location to take a sample of the grain 104 stored within.

The mainframe 112 may be any size, shape, and configuration in order to provide a platform for the probe system 110 and allow a truck 101 to drive within and through the mainframe 112 and underneath the probe system 110. In some embodiments, the mainframe includes stanchions 113 that are securely attached to the ground (e.g., through footings or the like) to provide a foundation for the upper elements of the rigid chain probe system 110. The stanchions reach upwardly to a height that supports left and right girders 114 and front and rear crossbeams 115. The left and right girders 114 may be attached to the stanchions 113 in any way known in the art and reach from the rear of the autonomous grain processing facility 100 to the front. In an embodiment, the front and rear crossbeams 115 reach from one side of the autonomous grain processing facility 100 to the other. In some embodiments, the front and rear crossbeams 115 are attached to stanchions 113 in any fashion known in the art. In other embodiments, the front and rear crossbeams 115 are attached to the left and right girders 114 in any fashion known in the art. The embodiments shown in FIGS. 1A-1B shows only front and rear crossbeams 115 to allow for maximum movement of the subframe 120, although it should be known that more crossbeams may be used to add additional structure to the mainframe while making other allowances for the ability to change the fore/aft location of the probe system 110 (e.g., adjusting the location of the truck itself and/or any other adjustment) without deviating from the scope of the disclosure.

In an embodiment, the stanchions 113, the girders 114, and the crossbeams 115 cooperate to form a structure that supports the rigid chain probe system 110 and allows for a truck 101 to travel within the mainframe 112. The stanchions 113 may be tall enough to provide the height necessary for a truck 101 to drive underneath the left and right girders 114, the front and rear crossbeams 115, the subframe 120 and the probe system 110. In other embodiments, the stanchions 113 may provide some of the height necessary while the shape and configuration of the crossbeams 115 provide the height and width (e.g., by shaping the crossbeams 115 in an arch shape, a triangle shape, in a unitary formation, by attaching multiple beams together to form the beam, or any other combination of stanchions 113, girders 114, and crossbeams 115 to accomplish the support of the probe system 110 and space for the truck 101 to pass through) without deviating from the scope of the disclosure.

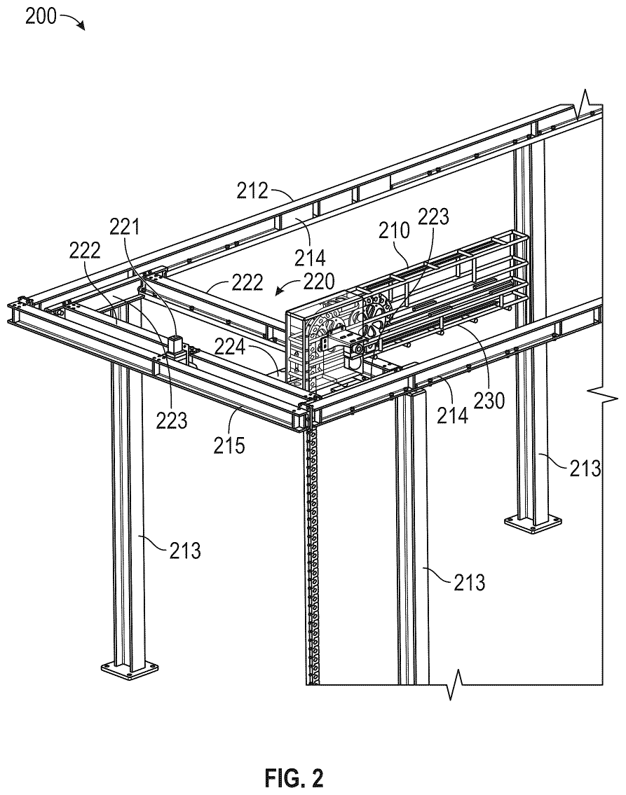

Looking now at FIG. 2, in some embodiments, the autonomous grain processing facility 200 is similar to the grain facility 100 from FIGS. 1A-1B except as described below, and may include a subframe 220 moveably attached to a mainframe assembly 212. The subframe 220 may be any size, shape, and configuration in order to provide a moveable but stable platform for the probe system 210 (e.g., via the chassis 230) to attach. The subframe 220 may include front and rear crossarms 222, left and right slidable beams 223, a support plate 224, and one or more motors 221. In other embodiments, more or fewer support beams or crossarms may be used to facilitate a moveable yet stable platform for probe system 210 (e.g., another set of crossarms and slidable beams similar to those described above).

In an embodiment, the subframe 220 attaches to the mainframe assembly 212 by slidably attaching left and right slidable beams 223 along the left and right girders 214. This attachment may be any attachment method known in the art (e.g., slidable attachment between the members, a rolling attachment through bearing, or any other attachment known in the art) that allows the slidable beams 223 to attach to and be movable along the girders 214. The slidable beams 223 may be moved by a motor 221 or by manually pushing the subframe to the desired location, either directly or through a pushing or pulling system (not shown).

In some embodiments, the subframe 220 includes front and rear crossarms 222 that reach between the slidable beams 223 at a front portion and a rear portion of the subframe 220. In such embodiments, as the slidable beams 223 are moved in the fore/aft direction, the crossarms 222 also move with the slidable beams 223. The crossarms 222 provide the connection, either directly or indirectly, for the probe system 210 to the subframe 220. In still other embodiments, the front and rear crossarms 222 are attached to the girders 214 and the slidable beams 223 are omitted. In still other embodiments, the front and rear crossarms 222 are directly attached to the girders 214 such that the subframe 220 cannot move in the fore/aft direction.

In some embodiments, the connection between the probe system 210 and the subframe 220 is through a support plate 224. The support plate may be any size, shape, and configuration suitable to attach to the front and rear crossarms 222, secure the probe system 210, and allow the probe to travel from the chassis 230, downwardly through the subframe 220, and into the grain 104 stored in the trailer of the truck 101. The support plate 224 may be a plate, a beam, a series of plates and/or beams, or any other elements known in the art to provide the benefits listed above. The support plate 224 may reach between and be supported by the front and rear crossarms 222, and include connections (e.g., fasteners, bolts, holes for fasteners to fit through, clips, pins, or any combination thereof known in the art) for the chassis 230 of the probe system 210 to securely attach to.

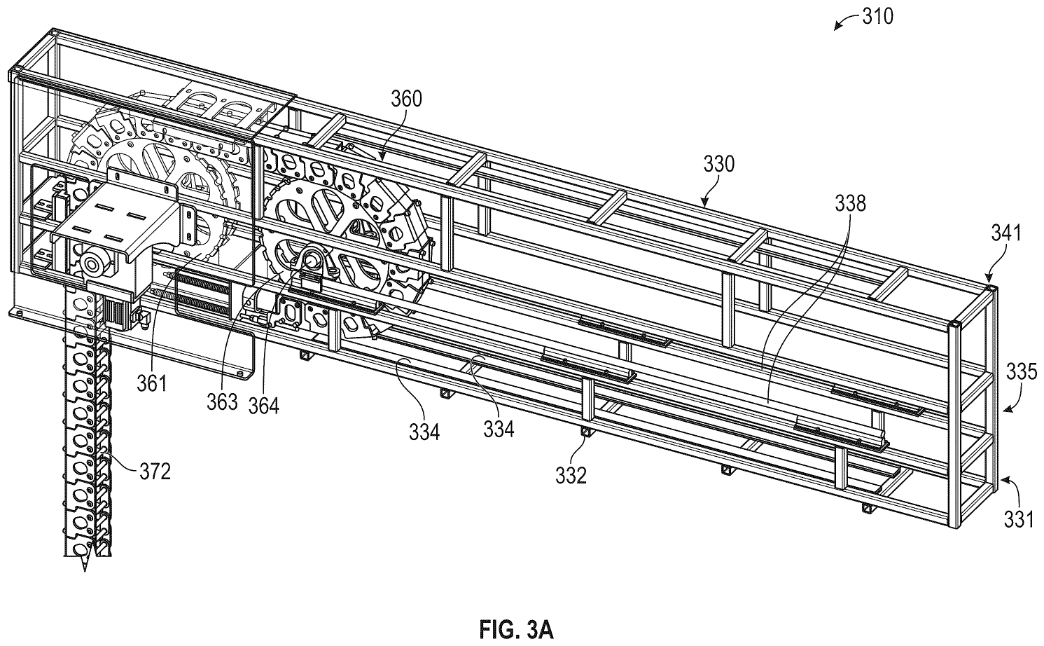

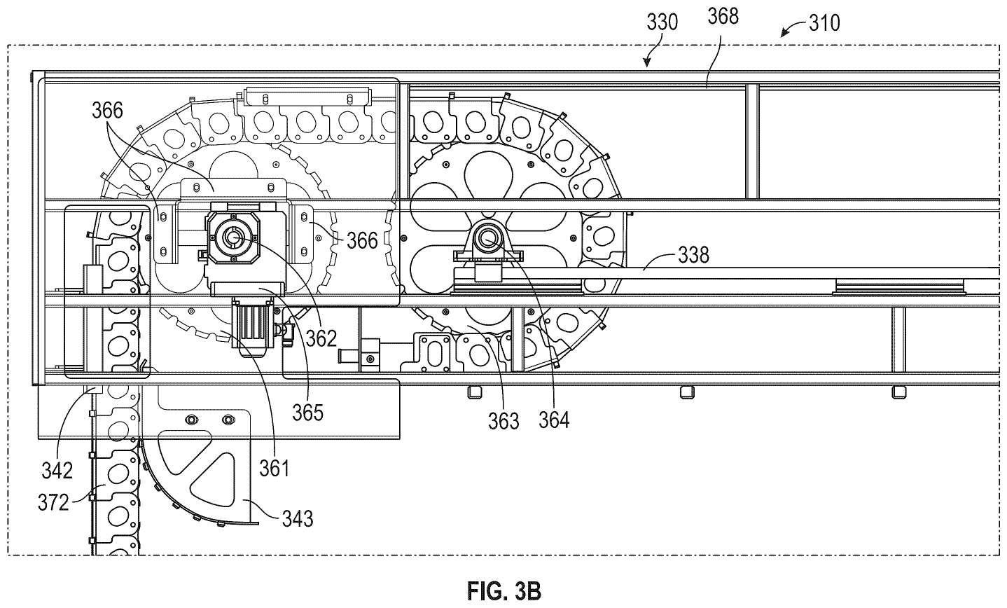

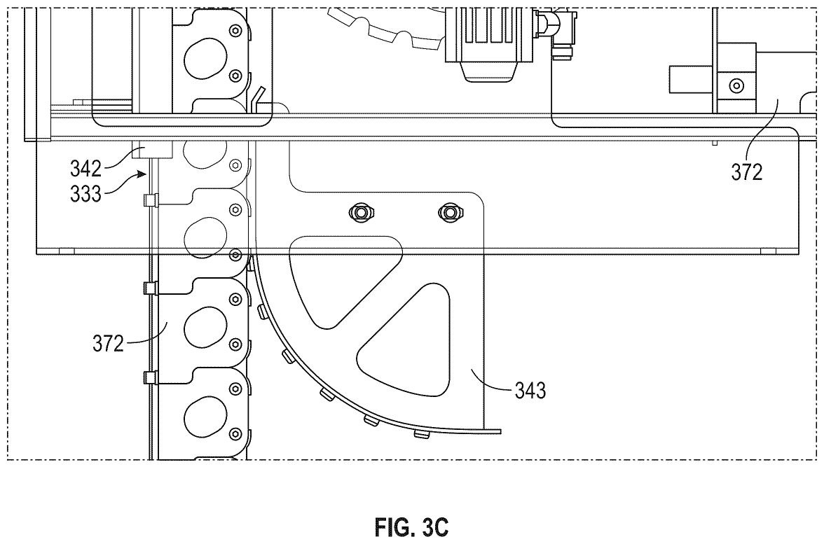

Turning now to FIGS. 3A-3C, in some embodiments, a rigid chain probe system 310 may be similar to rigid chain probe systems 110, 210 from FIGS. 1A-2 except as described below, and may include a chassis 330 that is attached to the moveable subframe 120 on the mainframe 112 (as shown and described above from FIGS. 1A-1B). The chassis may be any size, shape, and configuration suitable to provide a stable platform and space for containing the reel system 360. As shown in the figures, the chassis 330 is a substantially rectangular prism that has a longitudinal dimension that is greater than its height or side to side width, and has a height that is greater than its width. In some embodiments, the outer periphery of the chassis 330 is covered to protect internal workings of the chassis 330 such as the reel system 360. In other embodiments, the outer periphery of the chassis is uncovered to reduce the weight of the chassis 330.

The chassis 330 may be comprised of a number of steel tubes that are formed and connected in any fashion known in the art into the general shape of the chassis 330. As shown in FIGS. 3A-3C, in some embodiments, the chassis 330 includes horizontal support beams 332 that provide lateral support for the chassis 330. The horizontal support beams 332 may be placed at specific predetermined spots along a bottom side of the chassis 330, but leave an opening 333 near one end of the chassis for the probe 372 to reach downwardly through.

The chassis 330 may also include one or more horizontal rails 338. The horizontal rails may be any shape, size, and configuration suitable to provide a stable support for the reel sprocket 363 to slide along as the reel system 360 retracts and extends the probe 372 into the grain 104 in the trailer of the truck 101. As shown in FIGS. 3A-3C, the horizontal rails 338 are generally straight tubular steel rails attached to one or both sides of the chassis 330. In other embodiments, the reel sprocket 363 and its associated elements (such as but not limited to the horizontal rails 338, and axle bearing 364) may be removed and the excess chain length may simply fold upon itself within the chassis 330.

In some embodiments, the chassis includes a lower frame 331, a mid-frame 335, and an upper frame 341. The lower frame 331 can be any shape, size, and configuration suitable to provide the stable attachment of the chassis 330 to the subframe 120. The mid-frame 335 can be any shape, size, and configuration suitable to provide attachment (moveable and secure attachment) for and space for containment of the reel system 360. In some embodiments, the mid-frame 335 includes a slot within which the axle bearings 364 of the reel sprocket 363 can slide within and allowing the reel sprocket to slide fore and aft within the chassis 330. In other embodiments, the horizontal rail 338 and the axle bearing 364 are contained within the chassis 330 and do not need the slots of the mid-frame 335. The upper frame 341 can be any shape, size, and configuration suitable to provide protection of the internal workings of the chassis 330 such as the reel system 360.

Looking now at FIG. 3C, in some embodiments, the chassis 330 includes a breaking foot 342 and a probe relief quarter sprocket 343. The breaking foot 342 may be any size, shape, and configuration suitable to provide a strong and stable base upon which the probe 372 will snap cleanly if the probe 372 is extended into the grain 104 when the truck 101 (as shown and described above in FIGS. 1A-1B) moves backwardly, as will be described in more detail below. In the exemplary embodiment shown, the breaking foot 342 is a flat piece of sheet steel securely attached to the chassis 330 on the rear side of the opening 333. The probe relief quarter sprocket 343 may be any size, shape, and configuration suitable to provide a stable stress relief for the probe if the probe 372 is extended into the grain 104 and the truck 101 moves forwardly. In the exemplary embodiment shown, the probe relief quarter sprocket 343 is a stationary quarter sprocket that guides the probe 372 around an up to 90 degree turn from a downward direction to a forward direction without harming any elements of the probe 372 as described in detail below.

Staying in FIGS. 3A-3C, in some embodiments, a reel system 360 may be located substantially within the chassis 330. The reel system may be any size, shape, and configuration suitable to extend and retract the probe 372 into and out of the grain 104 stored in a trailer of a truck 101. As shown in FIGS. 3A-3C, the reel system 360 includes a drive sprocket 361 on an axle 362, a reel sprocket 363 attached to the horizontal rails 338 through an axle bearing 364, a motor 365, and motor protection flanges 366. As shown in FIGS. 3A-3C, the reel system 360 includes a drive sprocket that rotates about a stationary (stationary in this case means that the axle does not translate up/down, fore/aft, or laterally, but may rotate) axle 362 and is driven by motor 365. As the drive sprocket 361 is rotated, the probe 372 is moved which in turn drives the reel sprocket 363 about movable (moveable in this case means that the axle 362 may move in a fore/aft direction in addition to rotation) axle bearings 364.

With one end of the probe 372 fixed to the chassis 330, the rotation of the drive sprocket 361 drives both a rotational and a translational movement of the reel sprocket 363 along the horizontal rail 338. In some embodiments, as the probe 372 is retracted into the chassis 330, the reel sprocket 363 is moved translationally rearward (i.e., an increasing distance from the drive sprocket) whereby the probe 372 is increasingly contained within the chassis 330, and as the probe 372 is extended, the reel sprocket 363 is moved translationally forward (i.e., a reducing distance from the drive sprocket) whereby the probe is extended downwardly into the grain 104. In other embodiments, the general direction of movement may be reversed (e.g., the reel sprocket 363 moves forward away from the drive sprocket) without deviating from the scope of the disclosure. In some embodiments, the chassis 330 may include lubricating strips 334 along the path of the probe 372 to lubricate the movable parts (as described in detail below) of the probe 372 as it passes through the chassis 330.

In still other embodiments, the reel sprocket 363 is omitted from chassis 330, along with the horizontal rail 338 and the bearing 364, and the links of the probe 372 are simply allowed to gather within the chassis 330. To aid in keeping the probe cleanly stored within the chassis 330, one or more magnets 368 may be disposed on a top side of the chassis 330 that hold that probe 372 along a top side of the chassis while it is being extend and/or retracted, while allowing sliding motion necessary to gather the links of the probe 372 within the chassis 330.

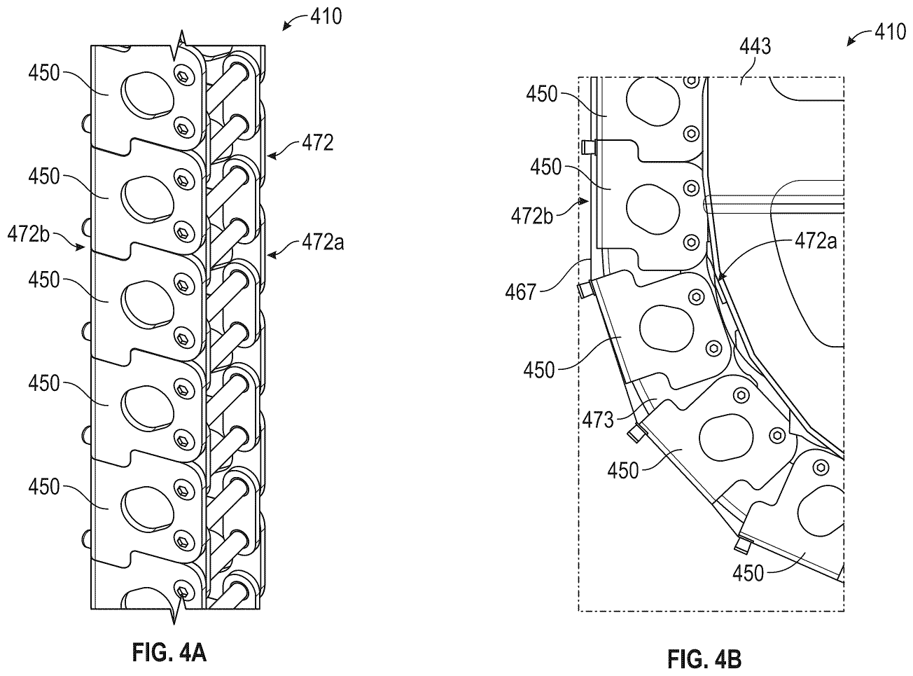

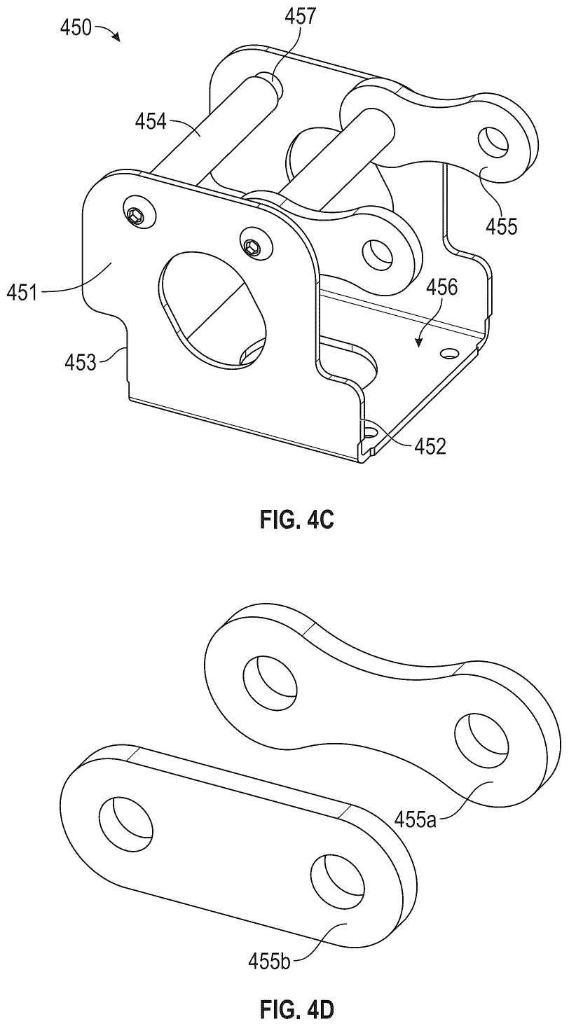

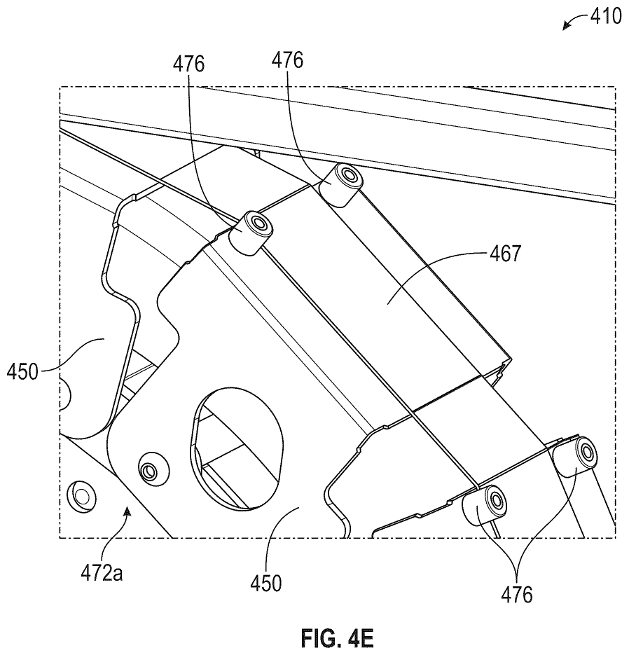

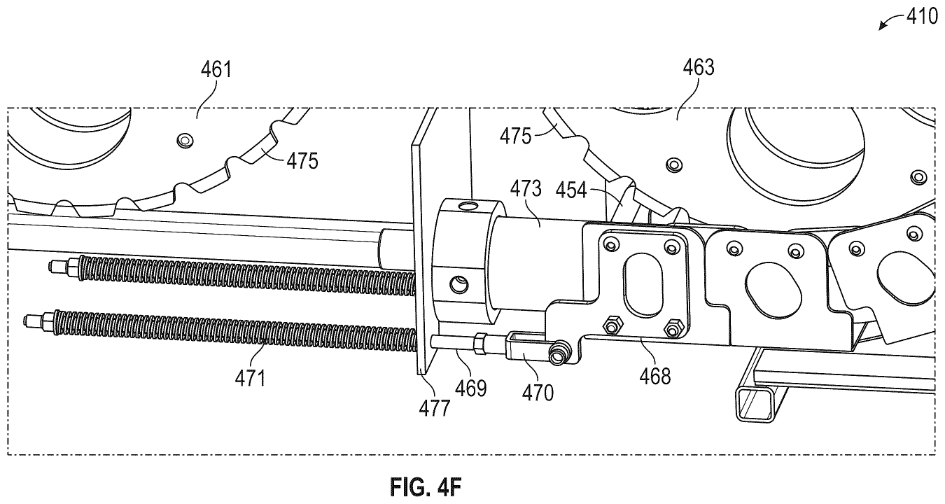

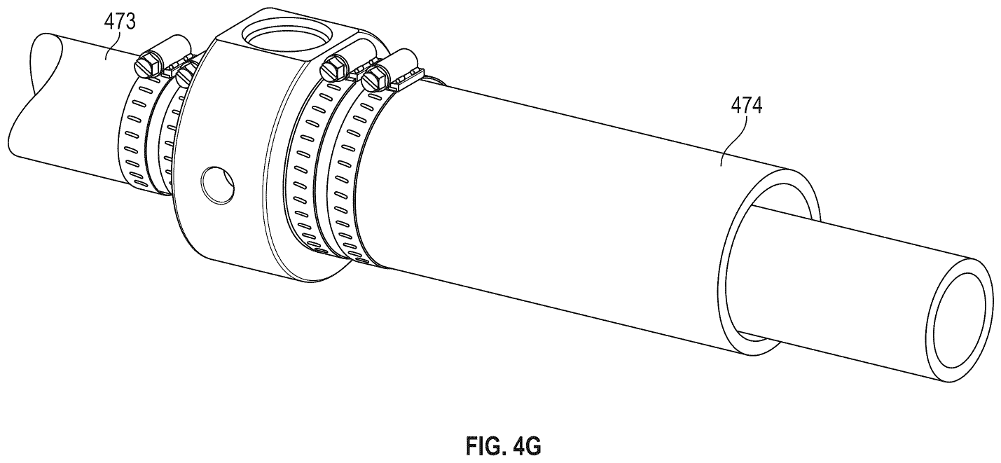

Looking at FIGS. 4A-4G, a rigid chain probe system 410 may be similar to rigid chain probe systems 110, 210, and 310 from FIGS. 1A-3C except as described below, and may include a probe 472. The probe 472 may be any size, shape, and configuration suitable to be reeled in and contained within a chassis 330 (as shown in FIGS. 3A-3C), extended into grain 104 (as shown in FIG. 1A) to be processed, and provide the strength and flexibility to remain intact if a truck 101 (as shown in FIG. 1A) begins pulling forward while the probe 472 is extended into the grain 104. As shown in FIGS. 4A-4G, the probe 472 may include a number of chain links 450, a tensioning cable or strap 467, a tensioning link 468, at least one tensioning bolt 469, a connector 470, at least one tensioning spring 471, a probe transfer hose 473, and a probe tip 474, although more or fewer elements of the probe 472 may be included without deviating from the scope of the disclosure.

As shown in FIGS. 4A-4B, in some embodiments, the probe 472 is made of a number of chain links 450 that are interconnected to guide and protect the transfer hose 473. The interconnected chain links 450 may be any size, shape, and configuration suitable to surround the transfer hose 473, to bend in one direction (e.g., in a direction as shown in FIG. 4B) at a radius safe for the transfer hose 473, and to resist bending in the opposite direction.

Looking at FIG. 4C, each chain link 450 may include a body 451 having an anti-rotation protrusion 452, a rotation indentation 453, and a center void 456, one or more drive pins 454, and one or more linkages 455. In some embodiments, the anti-rotation protrusion 452 of one chain link 450 cooperates with a rotation indentation 453 of an adjacent chain link 450 to allow rotation of the probe 472 in one direction, and prevent rotation of the probe 472 in the opposite direction. In some embodiments, the center void 456 is defined by the interior surface of the body 451 on three sides and the drive pins 454 on the fourth side, and is sized suitably to allow the transfer hose 473 of the probe 472 to fit within and be protected collectively by the bodies 451 of the each chain link 450.

The drive pins 454 may be any size, shape, and configuration suitable to provide lateral strength to the body 451 of the chain link 450, provide an attachment for the linkages 455 between adjacent chain links 450, and engage teeth 475 of the drive sprocket 461 and the reel sprocket 463. As shown in FIG. 4C, each drive pin may by substantially cylindrical and reach from one side of the body 451 to the other. The drive pin may further include an indent 457 for attachment and to prevent translational movement of the linkage 455 along the drive pin 454.

Looking at FIG. 4D, the linkage 455 may be any size, shape, and configuration suitable to connect adjacent chain links 450 and to keep the inside 472a of the chain links 450 of the probe 472 a constant distance apart. Keeping the inside 472a constant keeps the drive pins 454 a constant distance apart which allows the teeth 475 of the sprockets 461, 463 to appropriately engage the probe 472 for extending and retracting the probe 472. In the embodiments shown, the linkage 455 may be substantially obround with drive pin holes on either end. In some embodiments, the linkage 455a is scalloped for weight savings and a design weak point in the described case where a truck backs out of the parking spot while the probe is still extended. In other embodiments, the linkage 455b may not be scalloped for increased strength.

Turning to FIGS. 4E-4G, in some embodiments, the tensioning strap 467 may be any size, shape, and configuration suitable to provide tension to the outside 472b of the probe 472 such that the probe 472 is urged to resist the bending of the probe 472. In the exemplary embodiment shown, the rigid chain probe system 410 includes a tensioning strap 467, a tensioning link 468, a tensioning bolt 469, a connector 470, a spring 471, link guides 476, and a tensioning plate 477, although it should be known that more or fewer elements may be used to provide the tension necessary.

In some embodiments, the tensioning strap 467 is attached at a first end at the tensioning link 468 and wraps around the outside 472b of the probe 472 (as best shown in FIG. 4B) to the most distal chain link 450 on the probe 472. The tensioning strap is held to the body 451 of each chain link 450 by link guides 476 on the outside of each chain link 450 which prevents the tensioning strap from slipping off of the probe 472, while allowing longitudinal slippage of the strap as those portions of the probe 472 are rotated around the sprockets 461, 463. While the overall length of the outside of the probe 472 remains constant, the distance between the outside 472b of individual chain links 450 changes as they traverse the sprockets 461, 463, and the tensioning strap 467 needs to slide longitudinally along the outside of each chain link 450 as it does. In other embodiments, the link guides 476 may be eliminated and the tensioning strap 467 may simply slide on the back of each chain link 450. In still other embodiments, the tensioning strap 467 may be a cable that applies the tensioning force as described above as opposed to a flat strap without deviating from the scope of the disclosure. It should be known that the term strap or cable may be used interchangeably within the disclosure to accomplish this function.

In some embodiments as best shown in FIG. 4F, tension is applied to the tensioning strap 467 through one or more springs 471. The springs 471 are fitted within or around a bolt or other shaft 469 that is attached through a connector 470 to the tensioning strap 467. In some embodiments, the bolt 469 may be attached directly to the tensioning strap 467. In other embodiments, the bolts 469 are attached to a tensioning shaft 469a that the tensioning strap 467 attaches to as shown in FIG. 4G. The spring 471 is tensioned or squeezed between a head of the bolt 469 and a tensioning plate 477 and applies a constant tension to the strap which allows the downward extending portion to remain straight during use. The tensioning plate 477, in some embodiments, may also provide a connection for the transfer hose 473 of the probe 472. The springs 471 may be adjusted as a user desires by adjusting the length of the bolt 469, by changing the strength of the springs 471, or any other method of changing the tension on the tensioning strap 467 through the springs 471.

As shown in FIG. 4G, at a distal end of the probe 472, the transfer hose 473 may include a probe tip 474 for inserting into the grain 104 for processing. The transfer hose 473 may be connected to a vacuum source (not shown) in the facility 100 which allows a portion of the grain 104 to be pulled up from the truck 101 (as shown in FIGS. 1A-1B) and processed as desired. In still other embodiments, the hose 473 may include inner and outer hoses (not shown) and air may be urged through the outer hose to the tip and back through the inner hose which creates the negative pressure for the grain to be drawn up.

The use of elements of the various embodiments of the rigid chain probe systems 110, 210, 310, and 410 will be described with elements of the various embodiments used in conjunction with other embodiments, although it should be known that elements of each embodiment described may be added to or omitted without deviating from the scope of the disclosure. In use, the chassis 330 of the rigid chain probe system 110 may begin in a position on the mainframe 112 with the probe 472 in the retracted position (i.e., also with the reel sprocket 363 in a location distal from the drive sprocket 361 and a distal end of the probe 372 substantially within the chassis 330 as shown in FIG. 3B). As best shown in FIGS. 1A-1B, a truck 101 may drive into the mainframe 112 such that its trailer having trailer sections 105 is substantially within the mainframe 112 and then one of the section 105 is chosen for sampling.

Once a section and depth is determined for sampling, the subframe 120 may be moved to the desired sampling location of the trailer of the truck 101. The motor 365 is energized, rotating the drive sprocket (e.g., counterclockwise as shown in FIG. 3B) which extends the distal end of the probe 472 downwardly while simultaneously rotating the reel sprocket 363 and pulling it toward the drive sprocket 361, reducing the distance between the sprockets 361, 363. The tensioning strap 467 keeps tension on the outside 472b of the probe 472, which prevents the probe 472 from buckling or bending upon contact with the grain 104. Once the desired depth for the sample has been reached, the grain may be vacuumed up to the facility 100 for processing.

In the event that the truck 101 begins pulling forward while the probe 472 is still in the grain 104, the chain links 450 cooperate to bend the probe 472 around the relief quarter sprocket 443 (e.g., as shown in FIG. 4B) with the transfer hose 473 within such that the transfer hose 473 is not damaged. Once the trailer of the truck 101 has cleared the mainframe 112, the tensioning strap 467 pulls the probe 472 back to the straight position (e.g., as shown in FIG. 4A), and the probe 472 may be reeled back in.

In the event that the truck moves backward while the probe 472 is still in the grain 104, the chain links 450 are forced against the breaking foot 442 on the chassis 330. The probe 472 will snap off cleanly at the breaking foot 442, and a new length of chain links 450 and/or 455a and 455b may be quickly assembled and easily reattached to the probe 472.

When the probe 472 is desired to be reeled back in (e.g., once processing of the grain 104 is complete), the motor 365 is energized rotating the drive sprocket 361 (e.g., clockwise in FIG. 3B) which retracts the distal end of the probe 472 back up toward the chassis 330 while simultaneously rotating the reel sprocket 363 and urging it away from the drive sprocket 361, increasing the distance between the sprockets 361, 363. As the probe tip 474 is retracted into the chassis 330, excess length of the probe 472 is stored within the chassis 330 and is ready for the process to begin again.

Various examples of the disclosure have been described. Any combination of the described systems, operations, or functions is contemplated. These and other examples are within the scope of the following claims.

What is claimed is:

1. A probe support structure for driving a probe into granulate comprising:
a chassis comprising:
a lower frame configured to attach to a mainframe;
a midframe disposed substantially horizontally above the lower frame; and
a probe opening on a bottom portion of the chassis;
a reel assembly comprising:
a drive sprocket having an edge disposed above the probe opening; and
a motor disposed on the chassis and operably coupled to the drive sprocket;
a probe support disposed around the reel assembly, the probe support comprising:
a plurality of interlocking chain links, each chain link comprising:
an anti-rotation protrusion;
a rotation indentation on an opposite side of the chain link from the anti-rotation protrusion and configured to accept the anti-rotation protrusion from an adjacent chain link; and
at least one drive pin extending across a width of the chain link and configured to engage the drive sprocket such that movement of the drive sprocket drives the probe support; and
a probe disposed within a central space of each of the plurality of chain links and extending from a tensioning plate disposed on the chassis at a first end and through the link most distal from the tensioning plate at a second end.

2. The probe support of claim 1, wherein the anti-rotation protrusion and the rotation indentation cooperate to allow rotation of a first of the plurality of chain links with respect to a second of the plurality of chain links in one direction.

3. The probe support of claim 2, wherein the anti-rotation protrusion and the rotation indentation cooperate to prevent rotation of a first of the plurality of chain links with respect to a second of the plurality of chain links in a second direction opposite the first direction.

4. The probe support of claim 1, wherein each chain link comprises a pair of drive pins disposed on an inner portion of the chain link and substantially parallel to one another.

5. The probe support of claim 1, further comprising at least one linkage connecting the at least one drive pin of a first chain link to the at least one drive pin of a second chain link adjacent to the first chain link.

6. The probe support of claim 5, wherein the at least one linkage comprises a pair of linkages connecting a first drive pin of a first chain link to a second drive pin of a second chain link adjacent to the first chain link.

7. The probe support of claim 1, wherein the probe support further comprises a tensioning cable, and wherein the tensioning cable is configured to urge the anti-rotation protrusion of a first chain link and the rotation indentation of a second adjacent chain link together.

8. The probe support of claim 1, wherein the probe support further comprises a tensioning cable, and wherein the tensioning cable is configured to urge the anti-rotation protrusion of a first chain link and the rotation indentation of a second adjacent chain link together, thereby providing a stiffening force to a vertical probing portion of the probe support.

9. The probe support of claim 1, wherein the chassis is configured to attach to a subframe of the mainframe, the subframe selectively movable in at least one of a fore/aft direction and a lateral direction of the mainframe.

10. The probe support of claim 1, wherein the probe support comprises:
a fully extended position wherein the probe support reaches downwardly substantially to a floor of a container; and
a fully retracted position wherein the probe support is substantially contained within the chassis.

11. The probe support of claim 1, wherein the probe support further comprises a tensioning strap disposed on an outside of each of the plurality of chain links, the tensioning strap attached at a first end to the tensioning plate and at a second end to a chain link most distal from the tensioning plate.

12. The probe support of claim 1, wherein:
the chassis further comprises a horizontal rail; and
the reel assembly further comprises a reel sprocket slidably coupled to the horizontal rail.

13. The probe support of claim 12, wherein the probe support comprises:
a fully extended position wherein the reel sprocket is adjacent the drive sprocket and the probe support reaches downwardly substantially to a floor of a container; and
a fully retracted position wherein the reel sprocket is in a position most distal from the drive sprocket and the probe support is substantially contained within the chassis.

14. A probe reel assembly configured to provide support for driving a probe into granulate and allow flexion of the probe in one direction, the probe reel assembly comprising:
a chassis configured to attach to a mainframe, the chassis having a lower frame with a probe opening and a midframe disposed substantially horizontally above the lower frame;
a drive sprocket disposed above the probe opening;
a motor operably coupled to the drive sprocket;
a probe support comprising a plurality of interlocking chain links disposed around and operably connected to the drive sprocket;
wherein each of the plurality of chain links includes an anti-rotation protrusion, a rotation indentation on an opposite side of the chain link from the anti-rotation protrusion, and at least one drive pin extending across a width of the chain link and configured to engage the drive sprocket such that movement of the drive sprocket drives the probe support;
wherein the rotation indentation of each chain link is configured to accept the anti-rotation protrusion from an adjacent chain link; and
a probe disposed within a central space of each of the plurality of chain links and extending from a tensioning plate disposed on the chassis at a first end and through the link most distal from the tensioning plate at a second end.

15. The probe reel assembly of claim 14 further comprising a horizontal rail proximate the midframe, and a reel sprocket slidably coupled to the horizontal rail.

16. The probe reel assembly of claim 15 wherein the horizontal rail is disposed on the chassis.

17. The probe reel assembly of claim 15 wherein the reel sprocket is disposed on the horizontal rail.

18. The probe reel assembly of claim 14, further comprising a tensioning cable disposed on an outside of each of the plurality of chain links, the tensioning cable attached at a first end to the tensioning plate and at a second end to a chain link most distal from the tensioning plate.

19. A granulate probing system comprising:
a mainframe configured to allow a tractor trailer to drive underneath an upper frame portion;
a subframe disposed on the upper frame portion, the subframe selectively movable in a fore/aft and lateral directions on the upper frame portion;
a chassis disposed on the subframe comprising:
a lower frame with a probe opening therein;
a midframe disposed substantially horizontally above the lower frame; and
a horizontal rail proximate the midframe;
a reel assembly comprising:
a reel sprocket slidably coupled to the horizontal rail;
a drive sprocket having an edge disposed above the probe opening; and
a motor disposed on the chassis and operably coupled to the drive sprocket;
a probe support disposed around the reel sprocket and the drive sprocket, the probe support comprising:
a plurality of chain links cooperating in an interlocking manner to form the probe support, wherein each chain link comprises:
an anti-rotation protrusion;
a rotation indentation on an opposite side of the chain link from the anti-rotation protrusion and configured to accept the anti-rotation protrusion from an adjacent chain link; and
at least one drive pin extending across a width of the chain link and configured to engage the drive sprocket such that movement of the drive sprocket drives the probe support; and
a probe disposed within a central space of each of the plurality of chain links and extending from a tensioning plate disposed on the chassis at a first end and through the link most distal from the tensioning plate at a second end.

20. The granulate probing system of claim 19, wherein the probe support further comprises a tensioning cable disposed on an outside of each of the plurality of chain links, the tensioning cable attached at a first end to the tensioning plate and at a second end to a chain link most distal from the tensioning plate.
